(19) United States
(12) Patent Application Publication
(10) Pub. No.: US 2022/0313363 A1
(43) Pub. Date: Oct. 6, 2022

(54) Optical System And Apparatus For Instrument Projection And Tracking

(71) Applicant: DM1 LLC, Cheyenne, WY (US)

(72) Inventor: Colin Brahmstedt, Boulder, CO (US)

(21) Appl. No.: 17/608,771

(22) PCT Filed: Jun. 23, 2020
(86) PCT No.: PCT/US2020/039058

§ 371 Date: Nov. 4, 2021

Related U.S. Application Data
(60) Provisional application No. 62/865,375, filed on Jun. 24, 2019.

(51) Int. Cl. A61B 34/20; A61B 8/08; A61B 90/00; G06T 3/00; G06T 7/70; G06T 19/00; G06T 7/00; G06T 7/20

(52) U.S. Cl. CPC A61B 34/20; A61B 8/0841; A61B 90/39; G06T 3/0093; G06T 7/70; G06T 19/006; G06T 7/0012; G06T 7/20; A61B 2034/2063; A61B 2034/2057; A61B 2090/3937; A61B 2090/3983; G06T 2207/10132; G06T 2207/30204; G06T 2207/30004

(57) ABSTRACT

A method and system may be used for tracking a medical instrument. The method may include capturing image data. The method may include capturing ultrasound data. The ultrasound data may be captured via an ultrasound probe. The method may include dewarping the image data. The method may include searching for a marker in the dewarped image data. If it is determined that the marker is found, the method may include extracting an identification. The method may include comparing fiducials with a known geometry. The method may include determining a pose. The method may include determining a location of the medical instrument relative to the ultrasound probe. The method may include overlaying a three-dimensional projection of the medical instrument onto the ultrasound data.

### SUMMARY

In an aspect, a method may be used for tracking a medical instrument. The method may include capturing image data by various sources. The method may include capturing ultrasound data. The ultrasound data may be captured via an ultrasound probe. The method may include dewarping the image data. The method may include searching for a marker in the dewarped image data. If it is determined that the marker is found, the method may include extracting an identification. The method may include comparing fiducials with a known geometry. The method may include determining a pose. The method may include determining a location of the medical instrument relative to the ultrasound probe, determining ultrasound data, obtaining an ultrasound image, or any combination thereof. The method may include overlaying a three-dimensional projection of the medical instrument onto the ultrasound data, the ultrasound image, or both.

Another aspect may include a system for tracking a medical instrument. The system may include an ultrasound probe, a camera, a marker, and a computing device. The ultrasound probe may be configured to capture ultrasound data. The camera may be coupled to the ultrasound probe. The camera may be configured to capture marker image data. The marker may be coupled to the medical instrument. The computing device may be configured to dewarp the image data. The computing device may be configured to search for the marker in the dewarped image data. The computing device may be configured to extract an identification. The computing device may be configured to compare fiducials with a known geometry. The computing device may be configured to determine a pose. The computing device may be configured to determine a location of the marker and medical instrument relative to the ultrasound probe, determine ultrasound data, obtain an ultrasound image, or any combination thereof. The computing device may be configured to overlay a three-dimensional projection of the marker and medical instrument onto the ultrasound data, the ultrasound image, or both.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a block diagram of a system for instrument projection and tracking in accordance with embodiments of this disclosure.

FIG. 2 is a block diagram of another system for instrument projection and tracking in accordance with embodiments of this disclosure.

FIG. 3A is a diagram of a front view of an ultrasound probe in accordance with embodiments of this disclosure.

FIG. 3B is a diagram of a side view of an ultrasound probe in accordance with embodiments of this disclosure.

FIG. 3C is a diagram of a top view of an ultrasound probe in accordance with embodiments of this disclosure.

FIG. 4A is a diagram of a front view of a camera in accordance with embodiments of this disclosure.

FIG. 4B is a diagram of a side view of a camera in accordance with embodiments of this disclosure.

FIG. 4C is a diagram of a top view of a camera in accordance with embodiments of this disclosure.

FIG. 5A is a diagram of a front view of a device in accordance with embodiments of this disclosure.

FIG. 5B is a diagram of a side view of a device in accordance with embodiments of this disclosure.

FIG. 5C is a diagram of a top view of a device in accordance with embodiments of this disclosure.

FIG. 5D is a diagram of an isometric view of a device in accordance with embodiments of this disclosure.

FIG. 6A is a diagram of an exploded view of the device shown in FIG. 5B.

FIG. 6B is a diagram of an exploded view of the device shown in FIG. 5C.

FIG. 6C is a diagram of an example of the device shown in FIGS. 5A-5C.

FIG. 7 is a diagram of an optical system for instrument projection and tracking in accordance with embodiments of this disclosure.

FIG. 8 is a diagram of a monitor display in accordance with embodiments of this disclosure.

FIGS. 9A to 9L are diagrams of example geometries for a marker in accordance with embodiments of this disclosure.

FIG. 10 is a diagram of an example image of a needle coupled to a marker in accordance with embodiments of this disclosure.

FIG. 11 is a flow diagram of a method for instrument projection and tracking in accordance with embodiments of this disclosure.

### DETAILED DESCRIPTION

Reference will now be made in greater detail to a preferred embodiment of the invention, an example of which is illustrated in the accompanying drawings. Wherever possible, the same reference numerals will be used throughout the drawings and the description to refer to the same or like parts.

Many medical procedures require the placement of a needle in order for a procedure to be performed. These procedures include, and are not limited to, central line access, peripheral venous access, peripheral nerve blocks, and core needle biopsies. For example, vessels near the surface of the skin can be easily seen, however in some cases, the target vessel is too deep to see from the surface giving the medical provider no indication of the position relative to the target vessel. The medical provider may be a physician, a physician assistant, a nurse, a nurse practitioner, or any other qualified medical personnel. In some cases, the medical provider may be a robot or robot assisted clinician. Ultrasound is a standard method to identify subsurface vessels and tissues for prospective needle placement in deep tissue. Ultrasound guidance provides a cross-section of the target. Using ultrasound guidance, care providers may obtain live feedback of the position of an instrument relative to the target location when the image of the needle becomes a data image from the ultrasound probe. Ultrasound guidance may reduce the risk of missing targeted tissue, potential complications, and increases the ability of a care provider to access previously inaccessible areas, however it cannot locate and track the tip of the needle position in real-time, either prior to skin insertion or during insertion prior to the needle being imaged by the ultrasound probe. If the provider advances the needle too deep, the ultrasound image will appear to indicate that the needle is placed correctly in the target vessel, when actually the needle has penetrated and passed through the intended target. Due to the limitations of a single two-dimensional plane ultrasound imaging system, it is difficult to co-locate the trajectory of an instrument and the target tissue or vessel both prior to skin insertion and after skin insertion.

Typical solutions use an electromagnetic field to track the instrument tip location. An external antenna that is placed near the patient emits an electromagnetic field. These solutions require that a sensor is placed in the tip of the instrument to be tracked. The sensor is connected to a device configured to resolve the orientation of the sensor in three-dimensional space via a wired connection. These solutions require a second sensor that is attached to the ultrasound probe to determine the orientation of the ultrasound probe. These solutions are expensive and require a large antenna field footprint. In addition, the components are non-disposable and require sterilization, which increases the risk of spreading infection. Having a wire connected to the instrument may block the functionality of the instrument. In addition, these solutions require the use of proprietary instruments, potentially increasing ongoing costs. Some solutions include a physical needle guide that may be clipped onto the ultrasound probe, however these solutions are impractical in use. The embodiments disclosed herein offer a low cost solution by providing the care provider one or more synchronized co-located optimal views of the target tissue and instrument. The embodiments disclosed herein are compatible with any existing ultrasound equipment and any medical instrument. The embodiments disclosed herein incur minimal disruption to standard operating procedures.

As used herein, the terminology “instrument” may be any device that may be used for ultrasound guided applications, including, but not limited to central venous cannulation, local/regional nerve block, cyst aspiration, fine needle aspiration (FNA), core needle biopsy, peripherally inserted central catheter (PICC) line placement, arterial line placement, peripheral venous cannulation, and radio frequency (RF) ablation. In some embodiments, the instrument may include a needle or any type of device that is configured for insertion into a patient.

As used herein, the terminology “computer” or “computing device” includes any unit, or combination of units, capable of performing any method, or any portion or portions thereof, disclosed herein.

As used herein, the terminology “processor” indicates one or more processors, such as one or more special purpose processors, one or more digital signal processors, one or more microprocessors, one or more controllers, one or more microcontrollers, one or more application processors, one or more central processing units (CPU)s, one or more graphics processing units (GPU)s, one or more digital signal processors (DSP)s, one or more application specific integrated circuits (ASIC)s, one or more application specific standard products, one or more field programmable gate arrays, any other type or combination of integrated circuits, one or more state machines, or any combination thereof.

As used herein, the terminology “memory” indicates any computer-usable or computer-readable medium or device that can tangibly contain, store, communicate, or transport any signal or information that may be used by or in connection with any processor. For example, a memory may be one or more read only memories (ROM), one or more random access memories (RAM), one or more registers, low power double data rate (LPDDR) memories, one or more cache memories, one or more semiconductor memory devices, one or more magnetic media, one or more optical media, one or more magneto-optical media, or any combination thereof.

As used herein, the terminology “instructions” may include directions or expressions for performing any method, or any portion or portions thereof, disclosed herein, and may be realized in hardware, software, or any combination thereof. For example, instructions may be implemented as information, such as a computer program, stored in memory that may be executed by a processor to perform any of the respective methods, algorithms, aspects, or combinations thereof, as described herein. Instructions, or a portion thereof, may be implemented as a special purpose processor, or circuitry, that may include specialized hardware for carrying out any of the methods, algorithms, aspects, or combinations thereof, as described herein. In some implementations, portions of the instructions may be distributed across multiple processors on a single device, on multiple devices, which may communicate directly or across a network such as a local area network, a wide area network, the Internet, or a combination thereof.

As used herein, the terminology “determine” and “identify,” or any variations thereof, includes selecting, ascertaining, computing, looking up, receiving, determining, establishing, obtaining, or otherwise identifying or determining in any manner whatsoever using one or more of the devices and methods shown and described herein.

As used herein, the terminology “example,” “embodiment,” “implementation,” “aspect,” “feature,” or “element” indicates serving as an example, instance, or illustration. Unless expressly indicated, any example, embodiment, implementation, aspect, feature, or element is independent of each other example, embodiment, implementation, aspect, feature, or element and may be used in combination with any other example, embodiment, implementation, aspect, feature, or element.

As used herein, the terminology “or” is intended to mean an inclusive “or” rather than an exclusive “or.” That is, unless specified otherwise, or clear from context, “X includes A or B” is intended to indicate any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from the context to be directed to a singular form.

Further, for simplicity of explanation, although the figures and descriptions herein may include sequences or series of steps or stages, elements of the methods disclosed herein may occur in various orders or concurrently. Additionally, elements of the methods disclosed herein may occur with other elements not explicitly presented and described herein. Furthermore, not all elements of the methods described herein may be required to implement a method in accordance with this disclosure. Although aspects, features, and elements are described herein in particular combinations, each aspect, feature, or element may be used independently or in various combinations with or without other aspects, features, and elements.

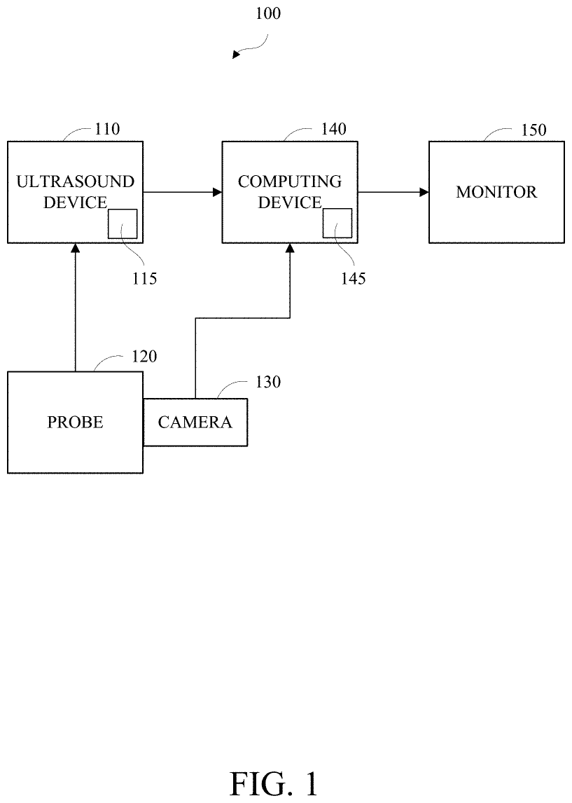

FIG. 1 is a block diagram of a system 100 for instrument projection and tracking in accordance with embodiments of this disclosure. As shown in FIG. 1, the system 100 includes an ultrasound device 110, a probe 120, a camera 130, a computing device 140, and a monitor 150.

The ultrasound device 110 includes a probe 120. The probe 120 may be a handheld probe. The probe 120 is configured to obtain a two-dimensional planar image of a portion of a patient. The probe 120 may be configured to use ultrasound, magnetic resonance, light, radio frequency (RF), or any other suitable diagnostic method capable of visualizing an internal portion of a patient. In this example, the probe 120 may communicate with the ultrasound device 110 via an ultrasound data cable. In some embodiments, the probe 120 may communicate with the ultrasound device 110 wirelessly, for example using any 802 technology, Bluetooth, near-field communication (NFC), or any other suitable wireless technology.

The probe 120 may be configured with a camera 130. The camera 130 may be removably attached to the probe 120, or it may be integrated with the probe 120. In some examples, the probe 120 may include two or more cameras. The camera 130 is configured to capture image data and send the image data to the computing device 140. The image data may be transmitted via a wired or wireless communication link. In an example, the camera 130 may be configured to rotate or flip such that the angle of the camera is adjustable and configurable by the user based on an angle of approach or user preference.

The ultrasound device 110 is configured to obtain ultrasound data via the probe 120. The ultrasound device 110 may include a processor 115 that is configured to process the ultrasound data and generate a video output. The ultrasound device 110 is configured to send the video output to the computing device 140. The ultrasound device 110 may transmit the video output via a wired or wireless communication link.

The computing device 140 is configured to receive the video output from the ultrasound device 110 and the image data from the camera 130. The computing device 140 may include a processor 145 that is configured to determine a position of a medical instrument (not shown) based on the image data from the camera 130 in real-time. The processor 145 of the computing device 140 may be configured to generate an overlay image that includes the determined position of the medical instrument in real-time. The processor 145 of the computing device 140 may be configured to merge the overlay image with the received video output from the ultrasound device 110 in real-time. The computing device 140 may be configured to overlay the positional information on the video stream and output the merged image to the monitor 150 for display in real-time. The computing device 140 may be configured to output the merged image in real-time via a wired or wireless communication link.

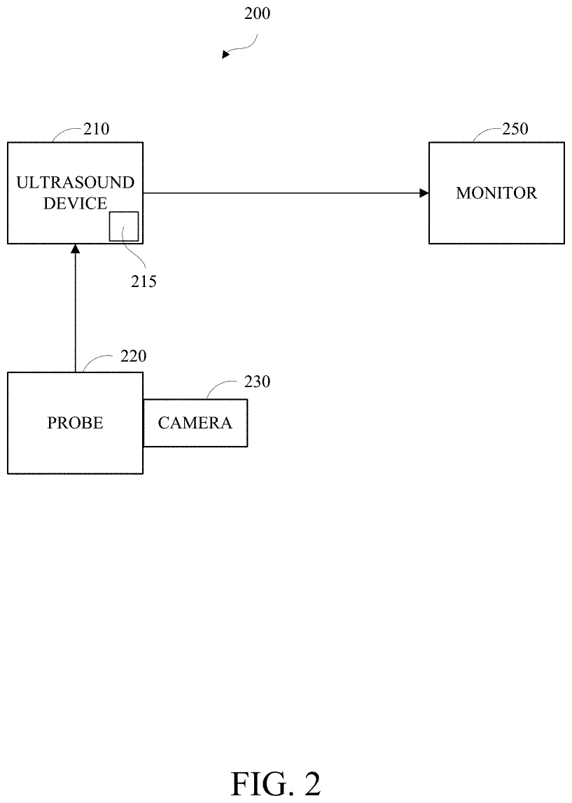

FIG. 2 is a block diagram of another system 200 for instrument projection and tracking in accordance with embodiments of this disclosure. As shown in FIG. 2, the system 200 includes an ultrasound device 210, a probe 220, a camera 230, and a monitor 250.

The ultrasound device 210 includes a probe 220. The probe 220 may be a handheld probe. The probe 220 is configured to obtain a two-dimensional planar image of a portion of a patient. The probe 220 may be configured to use ultrasound, magnetic resonance, light, radio frequency (RF), or any other suitable diagnostic method capable of visualizing an internal portion of a patient. the probe 220 may communicate with the ultrasound device 210 via an ultrasound data cable. In some embodiments, the probe 220 may communicate with the ultrasound device 210 wirelessly, for example using any 802 technology, Bluetooth, NFC, or any other suitable wireless technology.

The probe 220 may be configured with a camera 230. The camera 230 may be removably attached to the probe 220, or it may be integrated with the probe 220. In some examples, the probe 220 may include two or more cameras. The camera 230 is configured to capture image data and send the image data to the ultrasound device 210. The image data may be transmitted via a wired or wireless communication link. In an example, the camera 230 may be configured to rotate or flip such that the angle of the camera is adjustable and configurable by the user based on an angle of approach or user preference.

The ultrasound device 210 is configured to obtain ultrasound data via the probe 220. The ultrasound device 210 may include a processor 215 that is configured to process the ultrasound data and generate a video output. The ultrasound device 210 may be configured to receive the image data from the camera 230. The processor 215 may be is configured to determine a position of a medical instrument (not shown) based on the image data from the camera 230. The processor 215 of the ultrasound device 210 may be configured to generate an overlay image that includes the determined position of the medical instrument. The processor 215 of the ultrasound device 210 may be configured to merge the overlay image with the generated video output. The ultrasound device 210 may be configured to output the merged image to the monitor 250 for display. The ultrasound device 210 may be configured to output the merged image via a wired or wireless communication link.

In some examples, the camera 230 may include a processor (not shown) that is configured to determine a position of a medical instrument (not shown) based on the image data from the camera 230. The processor of the camera 230 may be configured to generate an overlay image that includes the determined position of the medical instrument and transmit the overlay image to the ultrasound device 210. The processor 215 of the ultrasound device 210 may be configured to merge the overlay image with the generated video output. The ultrasound device 210 may be configured to output the merged image to the monitor 250 for display. The ultrasound device 210 may be configured to output the merged image via a wired or wireless communication link.

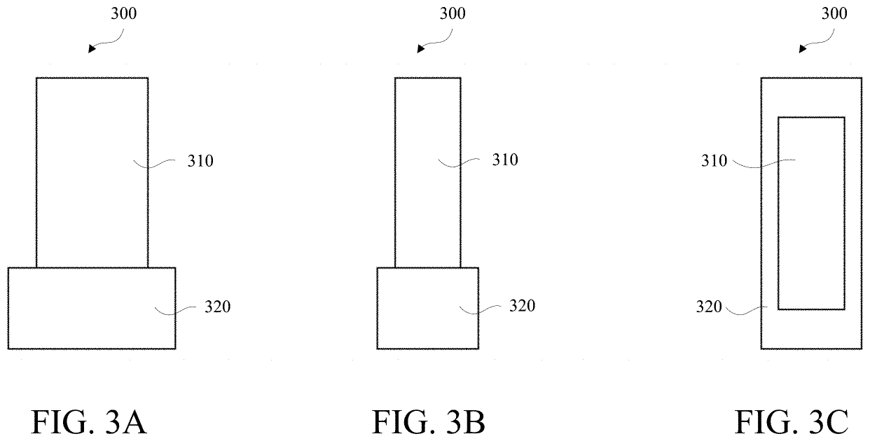

FIG. 3A is a diagram of a front view of an ultrasound probe 300 in accordance with embodiments of this disclosure. The view shown in FIG. 3A is along the long axis of the ultrasound probe 300. As shown in FIG. 3A, the ultrasound probe 300 includes a handle portion 310 and a sensor portion 320. The sensor portion 320 is configured to produce sound waves that reflect off body tissues and produce echoes. The sensor portion 320 includes a transducer that is configured to receive the echoes. The ultrasound probe 300 is configured to transmit the received echoes as ultrasound data to an ultrasound device, for example ultrasound device 110 of FIG. 1 or ultrasound device 210 of FIG. 2.

FIG. 3B is a diagram of a side view of an ultrasound probe in accordance with embodiments of this disclosure. The view shown in FIG. 3B is along the short axis of the ultrasound probe 300. As shown in FIG. 3B, the ultrasound probe 300 includes a handle portion 310 and a sensor portion 320. The sensor portion 320 is configured to produce sound waves that reflect off body tissues and produce echoes. The sensor portion 320 includes a transducer that is configured to receive the echoes. The ultrasound probe 300 is configured to transmit the received echoes as ultrasound data to an ultrasound device, for example ultrasound device 110 of FIG. 1 or ultrasound device 210 of FIG. 2.

FIG. 3C is a diagram of a top view of an ultrasound probe in accordance with embodiments of this disclosure. As shown in FIG. 3C, the ultrasound probe 300 includes a handle portion 310 and a sensor portion 320. The sensor portion 320 is configured to produce sound waves that reflect off body tissues and produce echoes. The sensor portion 320 includes a transducer that is configured to receive the echoes. The ultrasound probe 300 is configured to transmit the received echoes as ultrasound data to an ultrasound device, for example ultrasound device 110 of FIG. 1 or ultrasound device 210 of FIG. 2.

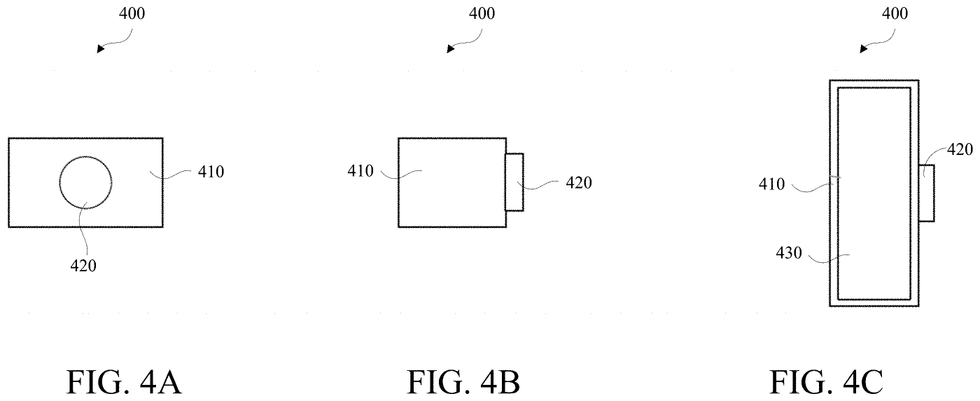

FIG. 4A is a diagram of a front view of a camera 400 in accordance with embodiments of this disclosure. As shown in FIG. 4A, the camera 400 includes a case 410 and a lens apparatus 420. The case 410 may be a detachable case that is configured to attach to one or more types of ultrasound probes. The case 410 may be a single-piece configuration, such as a sleeve or an interference fit, for example. In some embodiments, the case 410 may be a multi-piece configuration, such as a clamshell configuration, for example. The case 410 may include an opening that is configured to hold the lens apparatus 420. The lens apparatus 420 is configured to capture image data. In some examples, the case 410 may be configured to accommodate two or more cameras. In an example, the lens apparatus 420 may be configured to rotate or flip such that the angle of the lens apparatus 420 is adjustable and configurable by the user based on an angle of approach or user preference.

FIG. 4B is a diagram of a side view of a camera 400 in accordance with embodiments of this disclosure. As shown in FIG. 4B, the camera 400 includes a case 410 and a lens apparatus 420. The case 410 may be a detachable case that is configured to attach to one or more types of ultrasound probes. The case 410 may include an opening that is configured to hold the lens apparatus 420. The lens apparatus 420 is configured to capture image data.

FIG. 4C is a diagram of a top view of a camera 400 in accordance with embodiments of this disclosure. As shown in FIG. 4C, the camera 400 includes a case 410 and a lens apparatus 420. The case 410 may be a detachable case that is configured to attach to one or more types of ultrasound probes. The case 410 may include an opening that is configured to hold the lens apparatus 420. The case 410 includes a hollow portion 430 that is configured to attach to a handle of an ultrasonic probe. The hollow portion 430 may be configurable based on the dimensions of the handle of the ultrasonic probe. The lens apparatus 420 is configured to capture image data.

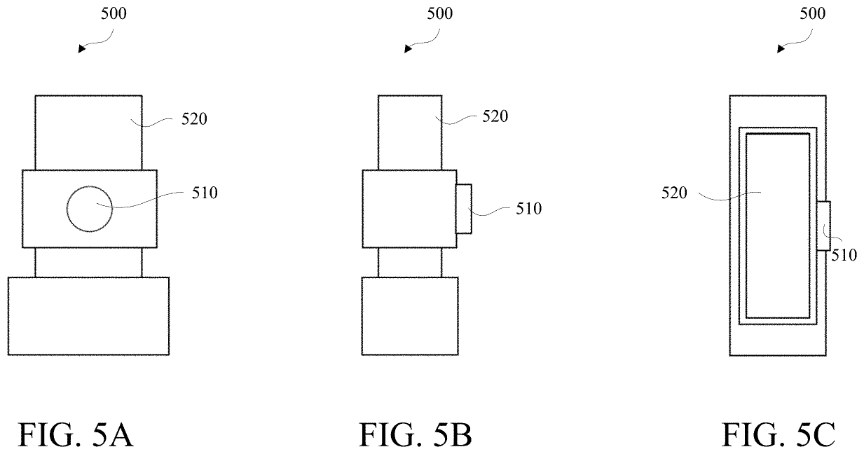

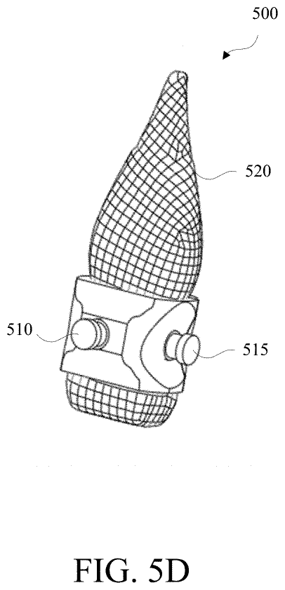

FIG. 5A is a diagram of a front view of a device 500 in accordance with embodiments of this disclosure. The device 500 includes a camera 510 coupled to an ultrasound probe 520. In some embodiments, the camera 510 may be integrated into the ultrasound probe 520. The camera 510 may be any camera, for example a detachable camera such as camera 400 shown in FIGS. 4A to 4C. The ultrasound probe 520 may be any ultrasound probe, for example ultrasound probe 300 shown in FIGS. 3A to 3C.

FIG. 5B is a diagram of a side view of the device 500 shown in FIG. 5A in accordance with embodiments of this disclosure. The device 500 includes the camera 510 coupled to the ultrasound probe 520. The camera 510 may be any camera, for example camera 400 shown in FIGS. 4A to 4C. The ultrasound probe 520 may be any ultrasound probe, for example ultrasound probe 300 shown in FIGS. 3A to 3C.

FIG. 5C is a diagram of a top view of the device 500 shown in FIG. 5A in accordance with embodiments of this disclosure. The device 500 includes a camera 510 coupled to an ultrasound probe 520. The camera 510 may be any camera, for example camera 400 shown in FIGS. 4A to 4C. The ultrasound probe 520 may be any ultrasound probe, for example ultrasound probe 300 shown in FIGS. 3A to 3C.

FIG. 5D is a diagram of the device 500 that includes two cameras. As shown in FIG. 5D, the device 500 includes a camera 510 and a camera 515 coupled to the ultrasound probe. The camera 515 may be used in long-axis approach procedures. The camera 510 may be any camera, for example camera 400 shown in FIGS. 4A to 4C. The camera 515 may be any camera, for example camera 400 shown in FIGS. 4A to 4C. The ultrasound probe 520 may be any ultrasound probe, for example ultrasound probe 300 shown in FIGS. 3A to 3C.

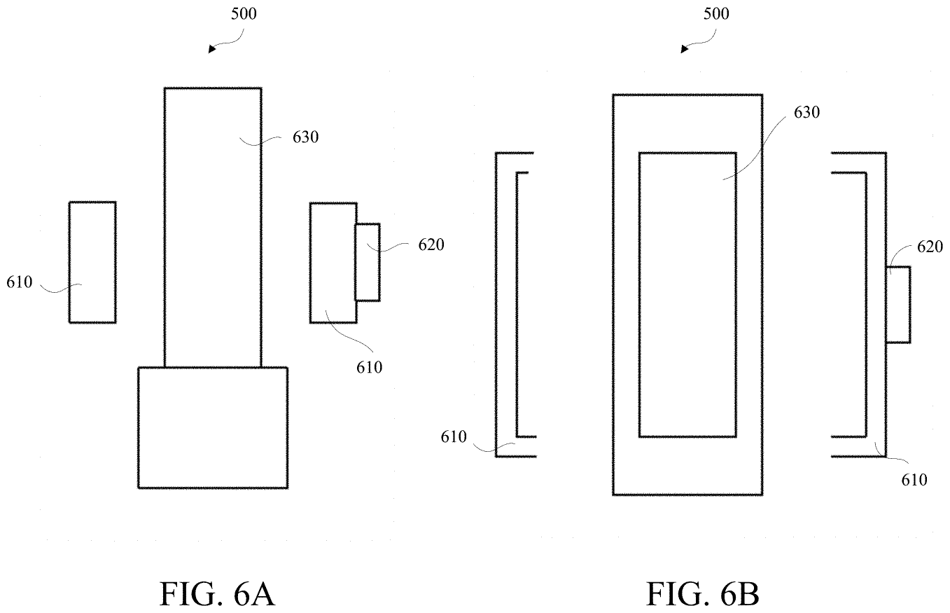

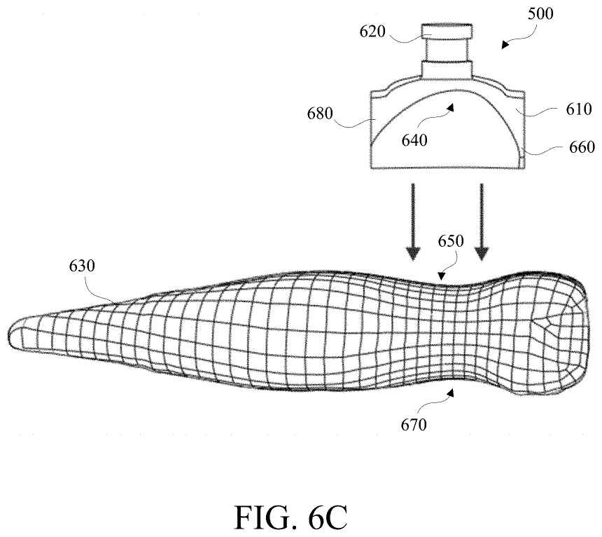

FIG. 6A is a diagram of an exploded view of the device 500 shown in FIG. 5B. As shown in FIG. 6A, the device 500 includes a case 610 and a lens apparatus 620. The case 610 may be a detachable case that is configured to attach to one or more types of ultrasound probes. In this example, the case 610 is shown in two parts, however the case 610 may be constructed of more than two parts. The case 610 may be in a single-piece configuration in some embodiments as described above. The case 610 may include an opening that is configured to hold the lens apparatus 620 of a camera. The case 610 includes a hollow portion that is configured to attach to a handle of an ultrasonic probe 630. The hollow portion may be configurable based on the dimensions of the handle of the ultrasonic probe 630.

FIG. 6B is a diagram of an exploded view of the device 500 shown in FIG. 5C. As shown in FIG. 6A, the device 500 includes the case 610 and the lens apparatus 620. The case 610 may be a detachable case that is configured to attach to one or more types of ultrasound probes. In this example, the case 610 is shown in two parts, however the case 610 may be constructed of more than two parts. The case 610 may include an opening that is configured to hold the lens apparatus 620 of a camera. The case 610 includes a hollow portion that is configured to attach to a handle of an ultrasonic probe 630. The hollow portion may be configurable based on the dimensions of the handle of the ultrasonic probe 630.

FIG. 6C is a diagram of an example of the device 500 shown in FIGS. 5A-5C. In this example, the device 500 includes the case 610 and the lens apparatus 620. The case 610 may be a detachable case that is configured to attach to one or more types of ultrasound probes. In this example, the case 610 is shown in a single part, such as an interference fit that is configured to clip onto the ultrasound probe 630. As shown in FIG. 6C, the case 610 includes an interior surface 640 that has a substantially same contour as an exterior surface 650 of the ultrasound probe 630. A side portion 660 of the case 610 may extend slightly around the rear portion 670 of the ultrasound probe 630 to provide a secure fit. In some examples, side portion 660 and side portion 680 may both extend slightly around the rear portion 670 of the ultrasound probe 630 to provide a secure fit.

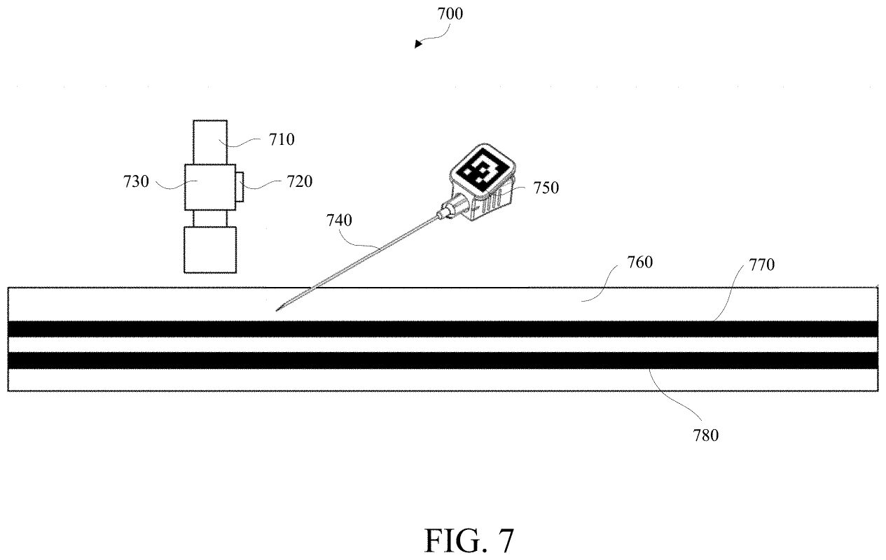

FIG. 7 is a diagram of an optical system 700 for instrument projection and tracking in accordance with embodiments of this disclosure. In this example, the optical system 700 includes an ultrasound probe 710. As shown in FIG. 7, a camera 720 is attached to the handle of the ultrasound probe 710. The camera 720 may be attached to the ultrasound probe 710 using a snap assembly 730, for example, a clam shell assembly shown in FIGS. 6A and 6B or an interference fit assembly shown in FIG. 6C.

The optical system 700 includes an instrument 740. In this example, the instrument 740 may be a needle. As shown in FIG. 7, the instrument 740 includes a marker 750. The marker 750 may be referred to as a fiducial. The marker 750 may be disposable. The marker 750 may be compatible with any luer lock instrument. In some embodiments, the marker 750 may be adapted for non-luer lock instruments via a snap, slip fit, sticker, or integrated into an instrument from the manufacturer. The marker 750 may be a detachable unit or a non-detachable unit. The marker 750 includes an identifier that may be captured by the camera 720 to identify the model of the instrument 740. The identifier may be a machine-scannable image such as a quick response (QR) code, barcode, or any other machine-scannable image. The identifier may include encoded data for the manufacturer and model of the attached instrument. The identifier may include encoded data associated with the marker, for example position data of the identifier. An example of the position data of the identifier may include an angle of the marker relative to the instrument. The identifier may be a sticker adhered to the marker, silkscreen printed directly on the marker, ultraviolet (UV) printed on the marker, or molded directly into the marker.

A body part 760 of a patient is shown as an example in FIG. 7. The body part 760 may be an arm, leg, groin, neck, abdomen, back, or any other body part. As shown in FIG. 7, the body part 760 includes a target vasculature 770 and a non-target vasculature 780. The instrument 740 is configured to enter the body part 760 and be placed in the target vasculature 770. The marker 750 is attached to the instrument 740 and is used to determine a three-dimensional position of the tip of the instrument 740. The marker 750 may also be used to track the trajectory, speed, and location of the tip of the instrument 740.

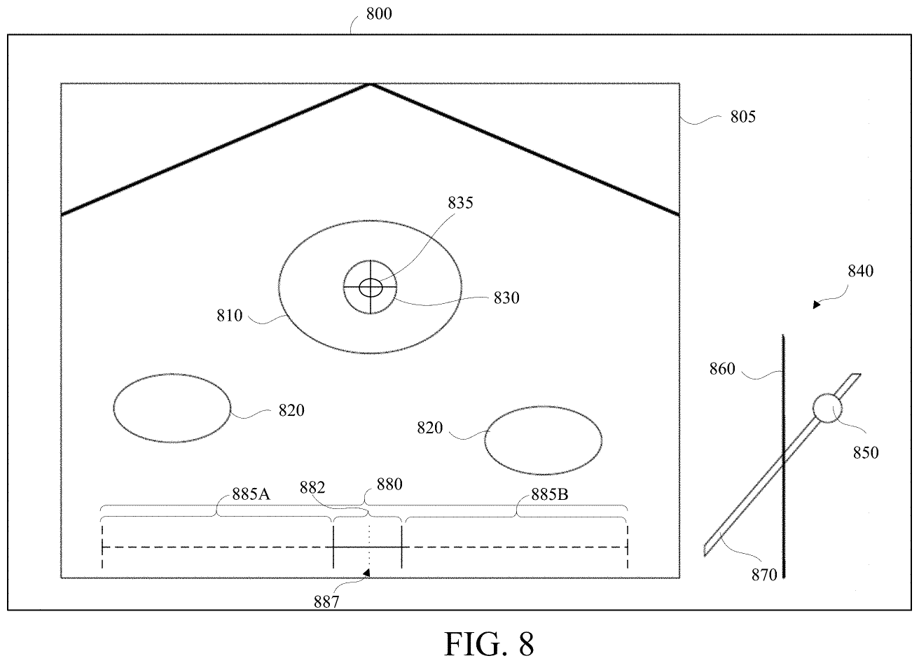

FIG. 8 is a diagram of a monitor display 800 in accordance with embodiments of this disclosure. As shown in FIG. 8, the monitor display 800 is an example of an ultrasound cross section view 805 using the system 700 shown in FIG. 7. The monitor display 800 shows a target vasculature 810 and non-target vasculature 820. A point 830 of an instrument is shown where it will intersect with the ultrasound cross section based on the current instrument trajectory. The point 830 may be displayed as an overlay on the monitor display 800, and may be displayed as cross-hairs as shown in FIG. 8. A point 835 shows the position of the instrument tip as it enters the target vasculature 810.

The monitor display 800 includes a projected side view 840 of the instrument trajectory. The projected side view 840 may show the distance between a current instrument position 850 and a side view of the plane of the ultrasound cross section 860. In this example, the current instrument position 850 may correspond with a tip of the instrument, for example the tip of a needle. The ultrasound cross section 805 is a front view of the ultrasound cross section 860. The projected side view 840 includes a trajectory 870 of the instrument.

The target area is shown as the point where the trajectory 870 and the ultrasound cross section 860 intersect. The current instrument projection 850 is used to track the depth of the tip of the instrument. The projected side view 840 may be used to determine if the current instrument position 850 passes the ultrasound cross section along the trajectory beyond the target area.

In another example, a depth gauge 880 may be displayed as an overlay on the monitor display 800. As shown in FIG. 8, the depth gauge 880 includes a target area 882, out-of-target areas 885A and 885B, and the current instrument tip position 887. In some examples, these areas may be depicted in colors, for example, the target area 882 may be shown in green or any suitable color, and the out-of-target areas 885A and 885B may be shown in red or any suitable color. The current instrument tip position 887 may be shown in any suitable color, for example, yellow. Movement of the target area is displayed in real-time, and the movement of the current instrument tip position 887 corresponds to the movement of the point 830. Movement of the current instrument tip position 887 along the depth gauge corresponds to the depth of the instrument tip. For example, when the current instrument tip position 887 is in the out-of-target area 885A, this would indicate that the instrument tip has not yet reached the depth of the target vessel 810, and the point 835 may not be visible. When the current instrument tip position 887 is in the out-of-target area 885B, this would indicate that the instrument tip has pierced through the target vessel. When the current instrument tip position 887 is in the target area 882, the point 835 is visible. Accordingly, when point 830 is aligned with the target vessel 810 and the current instrument tip position 887 is in the target area 882, this would indicate that the instrument tip is properly positioned in the target vessel 810.

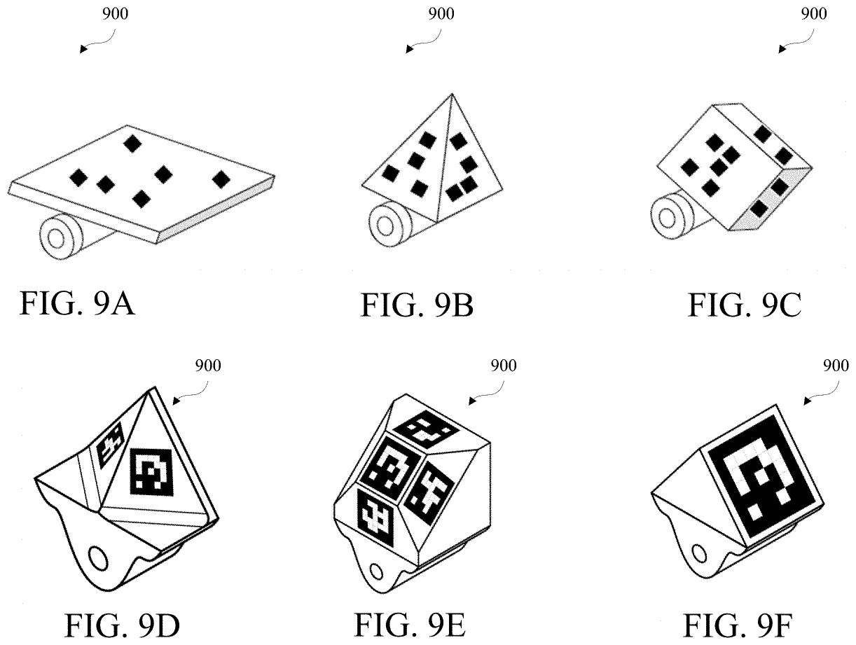

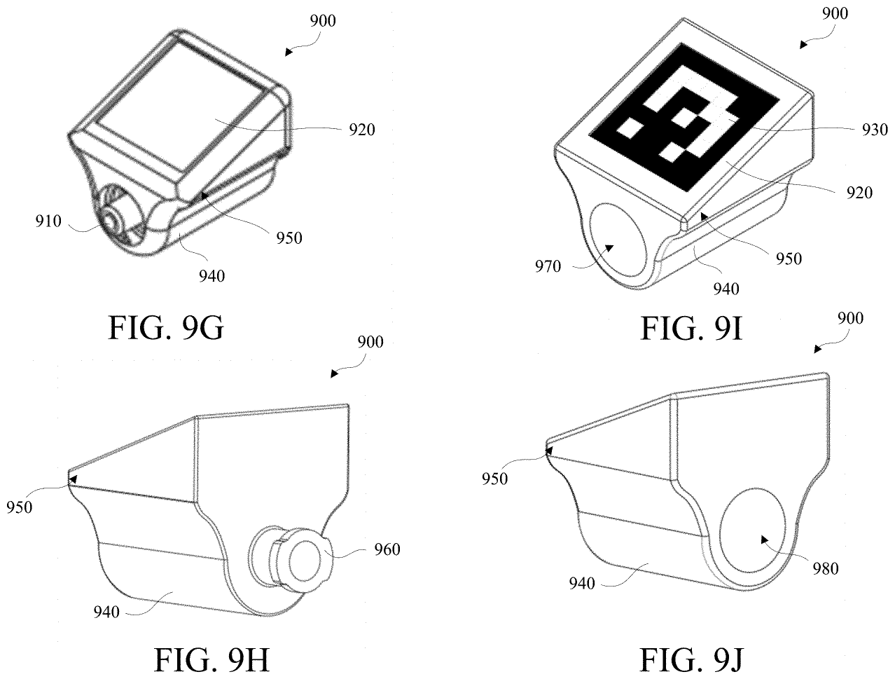

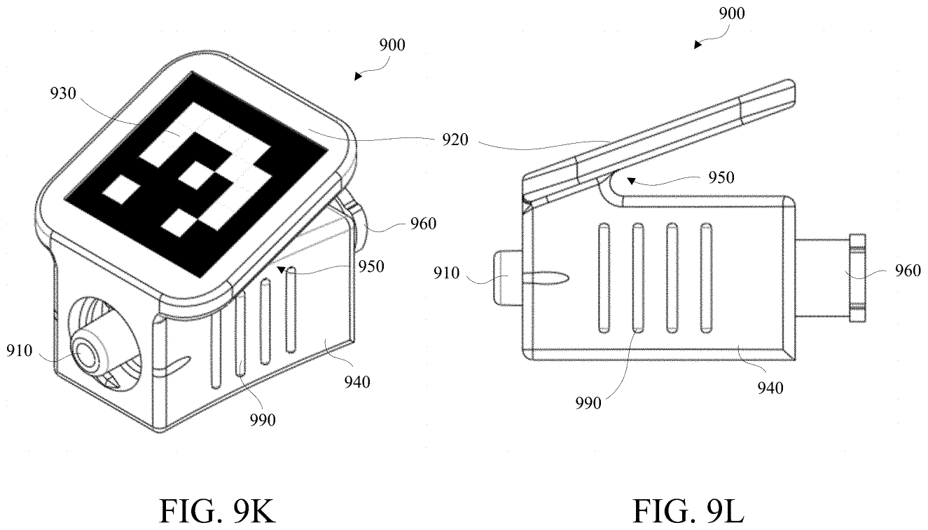

FIGS. 9A to 9I are diagrams of non-exhaustive example geometries 900 for a marker in accordance with embodiments of this disclosure. The marker may be marker 750 as shown in FIG. 7. Different geometries may be used based on the type and/or use of the instrument. FIG. 9A is a diagram of an example single-sided geometry for a marker. FIGS. 9B to 9F are non-exhaustive examples of multiple-sided geometries for a marker. The multiple-sided geometries may provide a benefit of improved precision based on more known trackable points, for example, from multiple markers. The markers may include encoded data for the manufacturer and model of the attached instrument.

FIGS. 9G and 9H are front and rear isometric views, respectively, of example geometries 900 for a marker in accordance with embodiments of this disclosure. As shown in FIG. 9G, the marker includes a fastener 910. The fastener 910 may be any fastener that can accommodate and attach an instrument such as a needle, catheter, or the like. In an example, the fastener 910 may be a luer lock type fastener. The marker includes a face 920 that includes an identifier 930, as shown in FIG. 9H. As shown in FIG. 9G, the face 920 may be attached to a base 940 at an angle 950 to enhance visibility of the face by a camera, such as camera 720 shown in FIG. 7. The angle 950 may be any angle between 0 degrees and 90 degrees. For example, the angle 950 shown in FIG. 9G is approximately 20 degrees. As shown in FIG. 9H, the marker includes a fastener 960. The fastener 960 may be any fastener that can accommodate and attach an instrument such as a syringe, or the like. In an example, the fastener 960 may be a luer lock type fastener.

FIGS. 91 and 9J are front and rear isometric views, respectively, of example geometries 900 for a marker with a slip fit configuration in accordance with embodiments of this disclosure. As shown in FIG. 9I, the marker includes an opening 970. The opening 970 may be configured to accommodate and attach an instrument such as a needle, catheter, or the like, such as a tapered handle of a shielded intravenous (IV) catheter. The opening 970 may have a diameter that ranges from approximately 7.0 cm to 10.5 cm. In an example, the opening 970 may have a diameter of about 9.3 cm. The marker includes a face 920 that includes an identifier 930. As shown in FIG. 9I, the face 920 may be attached to a base 940 at an angle 950 to enhance visibility of the face by a camera, such as camera 720 shown in FIG. 7. The angle 950 may be any angle between 5 degrees and 90 degrees. For example, the angle 950 shown in FIG. 9G is approximately 20 degrees. As shown in FIG. 9J, the marker includes an opening 980. The opening 980 may have a diameter that ranges from approximately 7.0 cm to 10.5 cm. In an example, the opening 980 may have a diameter of about 7.8 cm. The opening 970 may have a larger diameter than the opening 980 such that the internal taper of the openings matches the taper of a tapered handle of an instrument such that the marker may be slipped on and have a secure fit.

FIGS. 9K and 9L are front isometric and side views, respectively, of example geometries 900 for a marker in accordance with embodiments of this disclosure. As shown in FIG. 9K, the marker includes a fastener 910. The fastener 910 may be any fastener that can accommodate and attach an instrument such as a needle, catheter, or the like. In an example, the fastener 910 may be a luer lock type fastener. The marker includes a face 920 that includes an identifier 930. As shown in FIG. 9K, the face 920 may be attached to a base 940 at an angle 950 to enhance visibility of the face by a camera, such as camera 720 shown in FIG. 7. The base 940 may include one or more ridges 990. The one or more ridges 990 may be indented into the base 940, protruding from the base 940, or both. The one or more ridges 990 may enhance grip when attaching an instrument to the marker. Although the one or more ridges 990 are shown as parallel linear protrusions in FIGS. 9K and 9L, the one or more ridges 990 may be of any shape and arranged in any pattern, for example cross-hatching, circular dots, dimples, or the like. The angle 950 may be any angle between 5 degrees and 90 degrees. For example, the angle 950 shown in FIG. 9L is approximately 20 degrees. As shown in FIGS. 9K and 9L, the marker includes a fastener 960. The fastener 960 may be any fastener that can accommodate and attach an instrument such as a syringe, or the like. In an example, the fastener 960 may be a Luer lock type fastener.

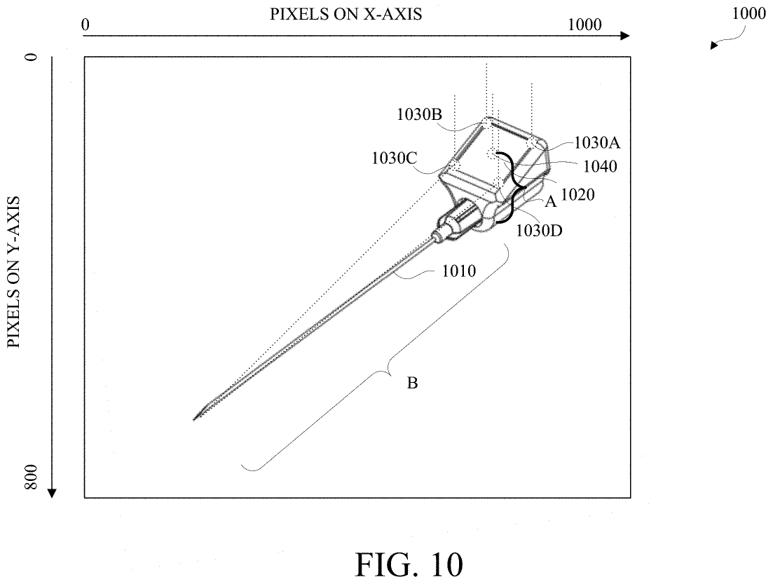

FIG. 10 is a diagram of an example image 1000 of a needle 1010 coupled to a marker 1020 in accordance with embodiments of this disclosure. A camera and software may be used to capture the image 1000, recognize the marker 1020, and resolve the location of a tip of an attached instrument, for example needle 1010, in three-dimensional space. The image 1000 may be scanned for features of a marker using methods including, but not limited to, Aruco, AprilTag, machine learning, or any combination thereof. The marker 1020 may be of any size or shape, and in this example may be a 15 mm by 15 mm square. The marker 1020 may be encoded with an identifier, such as an identification number, that indicates a manufacturer and model of the needle 1010. The identifier may also be used to identify which hub is being used, as each hub may have a different compatible needle associated with it. When the manufacturer and model of the needle 1010 is determined, the length of the needle may be determined. The length of the needle may be obtained from a look up table to determine the needle length, hub offset, or both. The software may be configured to project the tip of the instrument based on the marker location.

In this example, one or more of the points 1030A-D (shown in dotted lines) of the marker 1020 may be used as a reference for the software to determine the three-dimensional position of the marker 1020. The points 1030A-D may be referred to as fiducials of the marker. In this example, points 1030A-D are shown as the four corners of the marker 1020, however the points 1030A-D may represent any of the one or more points of the marker 1020 and not limited to the four corners. In this example, the marker 1020 may be a square marker, but the fiducials may be three-sided, such as in a triangle marker, to infinitely sided. The three-dimensional position of the marker 1020 may be used in conjunction with the identification of the marker 1020 to determine the location of the tip of the needle 1010.

As shown in FIG. 10, the image 1000 in this example may be approximately 1000 pixels along the x-axis and approximately 800 pixels along the y-axis. The image 1000 may be of any size, the pixel values along the x-axis and the y-axis are merely provided as examples. In this example, the camera may detect the marker 1020 and identify the points 1030A-D as fiducials. The location of each of the points 1030A-D may be determined as (x,y) pixel values, for example from the AprilTag library. Since the camera has identified marker 1020, it is known in this example that the marker is a 15 mm by 15 mm square. Based on the pixel values of the points 1030A-D, a processor of the camera, processor 115 of FIG. 1, or processor 145 of FIG. 1 may determine the best fit for how the marker 1020 is rotated and positioned in three dimensions to obtain a pose. The best fit may be determined using the solvePNPRansac method in OpenCV, for example.

A translation vector (tvec) and a rotational vector (rvec) may be determined. The tvec is associated with the (x,y,z) location of the center 1040 of the marker relative to the center of the camera. Z may be the distance away from the camera. The rvec may be associated with euler angles of how the marker 1020 is rotated along each of the axes, for example the x-axis may represent the pitch, the y-axis may represent the yaw, and the z-axis may represent the roll angle.

The processor of the camera, processor 115 of FIG. 1, or processor 145 of FIG. 1 may match the instrument, i.e. needle 1010 and determine the location of the needle tip. Once the marker has been identified, a lookup table may be used to determine the needle type that is attached to the marker 1020. When the needle type is determined, the dimensions of needle 1010 may be obtained from a lookup table. In an example, the distance (A) from the center 1040 of the marker to the needle body in the z-axis may be determined based on the marker identifier, and the distance (B) from the proximal end of the needle to the needle tip in the y-axis may be determined based on the needle type. The needle offset relative to the center of the marker 1020 may be determined based on the marker identifier. The location of the needle tip in a three-dimensional space and the pose/orientation relative to the center of the camera may be determined based on the distance A, distance B, needle offset, or any combination thereof.

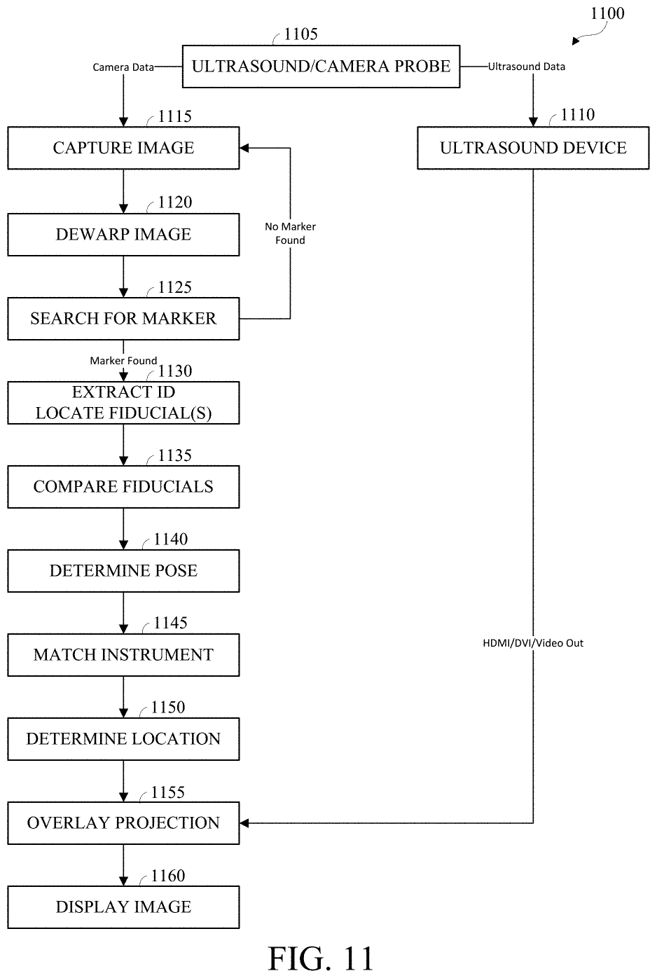

FIG. 11 is a flow diagram of a method 1100 for instrument projection and tracking in accordance with embodiments of this disclosure. As shown in FIG. 11, the ultrasound/camera probe 1105 is configured to send ultrasound data to the ultrasound device 1110 and camera data to a computing device (not shown). The ultrasound device 1110 may include an interface such as an high definition multimedia interface (HDMI) output, a digital visual interface (DVI) output, or any other video output to interface with the computing device. The computing device is configured to receive 1115 the camera data from the ultrasound/camera probe 1105. The computing device may be configured to dewarp 1120 the image to remove lens distortion. The computing device may be configured to search 1125 for a marker. If no marker is found, the computing device may return to receiving 1115 the camera data from the ultrasound/camera probe 1105. If a marker is found, the computing device may be configured to extract 1130 an identification and locate fiducials. In some embodiments, the computing device may not be present, and the functions of the computing device may be performed by the ultrasound device 1110.

The computing device may be configured to compare 1135 the fiducials with one or more previously known geometries. The computing device may be configured to determine 1140 a pose, for example, as discussed with reference to FIG. 10. The pose may be determined based on a translation of the rotation of the marker in a three-dimensional space. The pose may be based on the position of the fiducials represented in the two-dimensional camera image. The computing device may be configured to determine 1145 an instrument based on an identification embedded in the marker. The computing device may be configured to determine 1150 a location of the tip of the instrument relative to the ultrasound probe using a known three-dimensional model of the instrument. The computing device may be configured to overlay 1155 a three-dimensional projection of the instrument onto ultrasound data received from the ultrasound system 1110. The computing device may be configured to display 1160 the overlayed image. The overlayed image may be displayed on a separate display monitor or the monitor of the ultrasound system 1110.

Although some embodiments herein refer to methods, it will be appreciated by one skilled in the art that they may also be embodied as a system or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “processor,” “device,” or “system.” Furthermore, aspects of the present invention may take the form of a computer program product embodied in one or more computer readable mediums having computer readable program code embodied thereon. Any combination of one or more computer readable mediums may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to CDs, DVDs, wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks. The flowcharts and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method of tracking a medical instrument, the method comprising:
capturing image data;
capturing ultrasound data via an ultrasound probe;
dewarping the image data;
searching for a marker in the dewarped image data;
on a condition that the marker is found,
extracting an identification;
comparing fiducials with a known geometry;
determining a pose;
determining a location of the medical instrument relative to the ultrasound probe; and
overlaying a three-dimensional projection of the medical instrument onto the ultrasound data.

2. The method of claim 1, wherein determining the pose comprises translating the marker in a three-dimensional space based on a position of fiducials represented in the image data.

3. The method of claim 1 further comprising locating one or more fiducials.

4. The method of claim 1, wherein determining the location of the medical instrument comprises using a known three-dimensional model of the medical instrument.

5. The method of claim 1, wherein the known geometry is retrieved from a look up table.

6. A system for tracking a medical instrument, the system comprising:
an ultrasound probe configured to capture ultrasound data;
a camera coupled to the ultrasound probe, the camera configured to capture image data;
a marker coupled to the medical instrument; and
a computing device configured to:
dewarp the image data;
search for the marker in the dewarped image data;
extract an identification;
compare fiducials with a known geometry;
determine a pose;
determine a location of the medical instrument relative to the ultrasound probe; and
overlay a three-dimensional projection of the medical instrument onto the ultrasound data.

7. The system of claim 6, wherein the computing device is configured to determine the pose by translating the marker in a three-dimensional space based on a position of fiducials represented in the image data.

8. The system of claim 6, wherein the computing device is further configured to locate one or more fiducials.

9. The system of claim 6, wherein the computing device is configured to determine the location of the medical instrument by using a known three-dimensional model of the medical instrument.

10. The system of claim 6, wherein the computing device is configured to retrieve the known geometry from a look up table.

11. The system of claim 6, wherein the identification includes an instrument manufacturer, an instrument model, or both.

12. The system of claim 11, wherein the computing device is configured to determine a length of the medical instrument based on the instrument manufacturer, the instrument model, or both.

13. The system of claim 6, wherein the computing device is configured to determine the pose based on a position of one or more points of the marker.

14. The system of claim 6 further comprising a second camera coupled to the ultrasound probe.

15. The system of claim 14, wherein the second camera is disposed on a face of the ultrasound probe that is perpendicular to the camera.
